(19) United States
(12) Patent Application Publication
(10) Pub. No.: US 2008/0234831 A1
(43) Pub. Date: Sep. 25, 2008

(54) Bioresorbable Stent and Method of Making

(75) Inventors: Gerry Clarke, Gortachalla (IE); Hilda Mulvihill, Ballyclough (IE); Angela Duffy, Galway (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/688,427

(22) Filed: Mar. 20, 2007

(51) Int. Cl. A61F 2/04; B29C 39/00

(52) U.S. Cl. 623/23.7; 264/299

(57) ABSTRACT

A method of making a polymeric stent includes providing a molding apparatus including a central core pin and a plurality of slides, wherein each of the slides includes grooves on an inner surface the slide, wherein the grooves are formed in the shape of the stent. A molten polymer is injected into the grooves and allowed to solidify. The slides are moves away from the central core pin and the solidified polymer, in the shape of a stent, is removed. The central core also may or may not include corresponding grooves depending on the desired cross section of struts and crowns of the stent.

### FIELD OF THE INVENTION

The invention relates generally to a polymer stent and a method of making a polymer stent.

### BACKGROUND OF THE INVENTION

Stents have gained acceptance in the medical community as a device capable of supporting body lumens, such as blood vessels, that have become weakened or are susceptible to closure. Typically, a stent is inserted into a vessel of a patient after an angioplasty procedure has been performed to partially open up the blocked/stenosed vessel thus allowing access for stent delivery and deployment. After the catheter used to perform angioplasty has been removed from the patient, a tubular stent, maintained in a small diameter delivery configuration at the distal end of a delivery catheter, is navigated through the vessels to the site of the stenosed area. Once positioned at the site of the stenosis, the stent is released from the delivery catheter and expanded radially to contact the inside surface of the vessel. The expanded stent provides a scaffold-like support structure to maintain the patency of the region of the vessel engaged by the stent, thereby promoting blood flow. Physicians may also elect to deploy a stent directly at the lesion rather than carrying out a pre-dilatation procedure. This approach requires stents that are highly deliverable i.e. have low profile and high flexibility.

These non-surgical interventional procedures often avoid the necessity of major surgical operations. However, one common problem associated with these procedures is the potential release of embolic debris into the bloodstream that can occlude distal vasculature and cause significant health problems to the patient. For example, during deployment of a stent, it is possible for the metal struts of the stent to cut into the stenosis and shear off pieces of plaque which become embolic debris that can travel downstream and lodge somewhere in the patient's vascular system. Further, pieces of plaque material can sometimes dislodge from the stenosis during a balloon angioplasty procedure and become released into the bloodstream.

Various types of endovascular stents have been proposed and used as a means for preventing restenosis. A typical stent is a tubular device capable of maintaining the lumen of the artery open. One example includes the metallic stents that have been designed and permanently implanted in arterial vessels. The metallic stents have low profile combined with high strength. Restenosis has been found to occur, however, in some cases despite the presence of the metallic stent. In addition, some implanted stents have been found to cause undesired local thrombosis. To address this, some patients receive anticoagulant and antiplatelet drugs to prevent local thrombosis or restenosis, however this prolongs the angioplasty treatment and increases its cost.

A number of non-metallic stents have been designed to address the concerns related to the use of permanently implanted metallic stents. U.S. Pat. No. 5,984,963 to Ryan et al., discloses a polymeric stent made from resorbable polymers that degrades over time in the patient. U.S. Pat. No. 5,545,208 to Wolff et al. discloses a polymeric prosthesis for insertion into a lumen to limit restenosis. The prosthesis carries restenosis-limiting drugs that are released as the prosthesis is resorbed. The use of resorbable polymers, however, has drawbacks that have limited the effectiveness of polymeric stents in solving the post-surgical problems associated with balloon angioplasty.

Polymeric stents are typically made from bioresorbable polymers. Materials and processes typically used to produce resorbable stents result in stents with low tensile strengths and low modulus, compared to metallic stents of similar dimensions. The limitations in mechanical strength of the resorbable stents can result in stent recoil after the stent has been inserted. This can lead to a reduction in luminal area and hence blood flow. In severe cases the vessel may completely re-occlude. In order to prevent the recoil, polymeric stents have been designed with thicker struts (which lead to higher profiles) or as composites to improve mechanical properties. The use of relatively thick struts makes polymeric stents stiffer and decreases their tendency to recoil, but a significant portion of the lumen of the artery can be occupied by the stent. This makes stent delivery more difficult and can cause a reduction in the area of flow through the lumen. A larger strut area also increases the level of injury to the vessel wall and this may lead to higher rates of restenosis i.e. re-occlusion of the vessel. Thus, there exists a need for a bioresorbable stent with improved mechanical strength.

There also exists a need for an improved method of making a bioresorbable stent.

### BRIEF SUMMARY OF THE INVENTION

The present disclosure relates to a method of making a polymeric stent. A molding apparatus is provided including a central core pin and a plurality of slides, wherein each of the slides includes grooves on an inner surface of the slide, wherein the grooves are formed in the shape of the stent. A molten polymer is injected into the grooves and allowed to solidify. The slides are moved away from the central core pin and the solidified polymer, in the shape of a stent, is removed.

The outer surface of the central core pin also may or may not include corresponding grooves depending on the desired cross-section of struts and crowns of the stent. Without grooves on the outer surface of the central core pin, the cross-section of the struts and/or crowns of the stent will be D-shaped. With grooves on the outer surface of the central core pin, the cross-section will be rounded if grooves on the inner surface of the slides and the outer surface of the central core pin are round. The shape of the grooves can be selected to achieve the desired cross-section for the struts and crowns.

The present disclosure also relates to a bioresorbable polymer stent comprising three components: a drug; a bioresorbable polymer; and a bioresorbable glass fiber or particulate. The drug component is preferably an antiproliferative drug. The bioresorbable polymer may be any commonly used bioresorbable polymer. The bioresorbable glass fiber or particulate may be, for example, a bioactive hydroxyapatite based glass, such a Bioglass 45S5, or any other bioresorbable fiber that would improve the mechanical strength of the bioresorbable polymer. The three components are dissolved in a suitable solvent and the solution can then be processed into the desired stent shape by means such as injection molding, spraying, or casting.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a perspective view of an embodiment of an injection molding system for a stent in accordance with the present disclosure.

FIG. 2 is a partial cut-away view of the embodiment of FIG. 1 with the slides in a closed position.

FIG. 3 is a partial cut-away view of the embodiment of FIG. 1 with the slides in an open position

FIG. 4 is a cross-sectional view of strut of a stent of the present disclosure.

FIG. 5 is a cross-sectional view of a stent of the present disclosure mounted on a balloon.

FIG. 6 is a front elevation view of an alternative embodiment central core pin of an embodiment of an injection molding system for a stent in accordance with the present disclosure.

FIG. 7 is a perspective view of a polymer stent made in accordance with an embodiment of the present disclosure.

FIG. 8A is a plan view of the struts of a stent in accordance with another embodiment of the present disclosure.

FIG. 8B is a magnified view of a strut of the stent of FIG. 8A.

FIG. 9 is a cross-sectional view of the injection molding system of FIG. 1.

### DETAILED DESCRIPTION OF THE INVENTION

Specific embodiments of the present disclosure are now described with reference to the figures, wherein like reference numbers indicate identical or functionally similar elements. The present disclosure is directed to a method of making a polymer stent and a polymer stent.

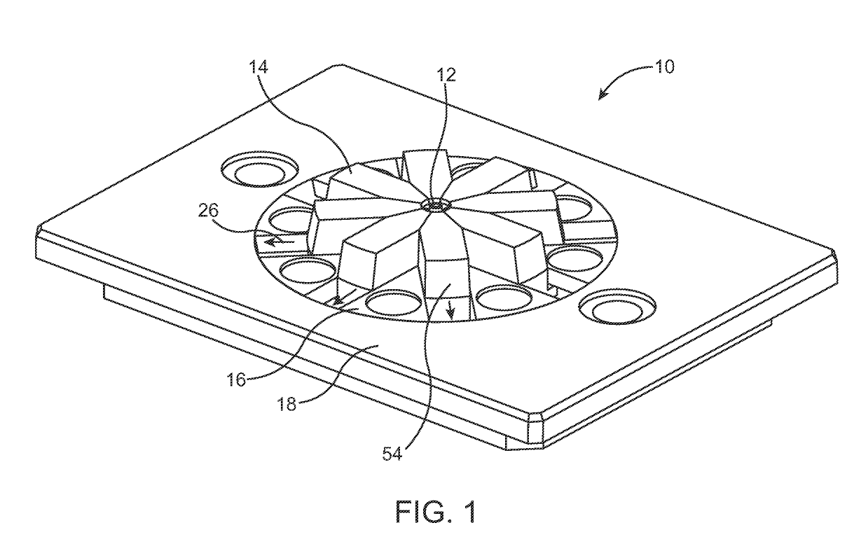

FIG. 1 is a perspective view of a rapid prototyping molding system 10 for forming a stent. The molding system 10 includes a base 18, slides 14, slide guide blocks 16, and a central core pin 12. FIG. 1 shows molding system 10 with slides 14 in a closed position. Slides 14 are moved to an open position by sliding them in the direction shown by arrows 26 in FIG. 1.

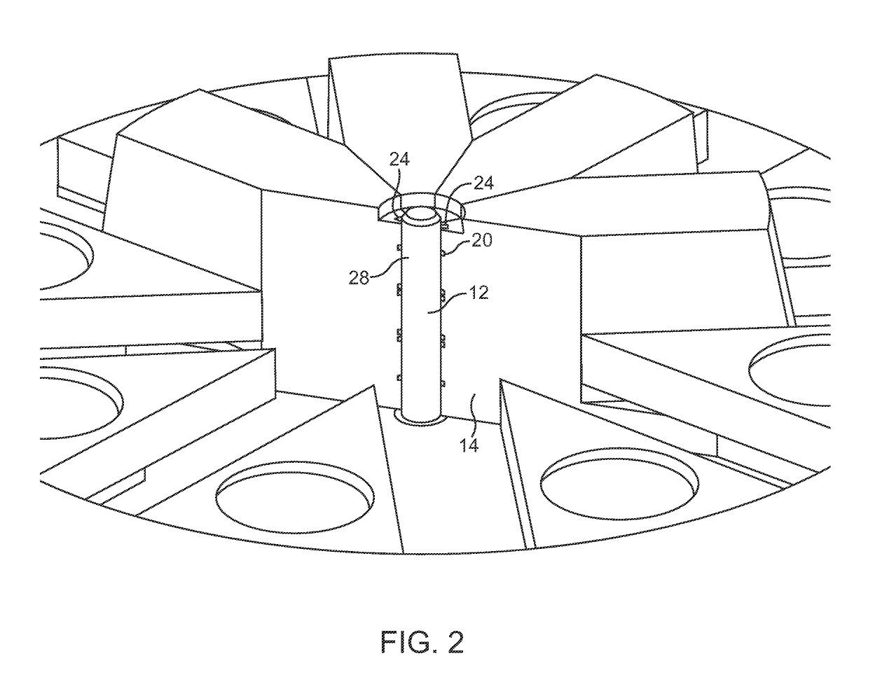

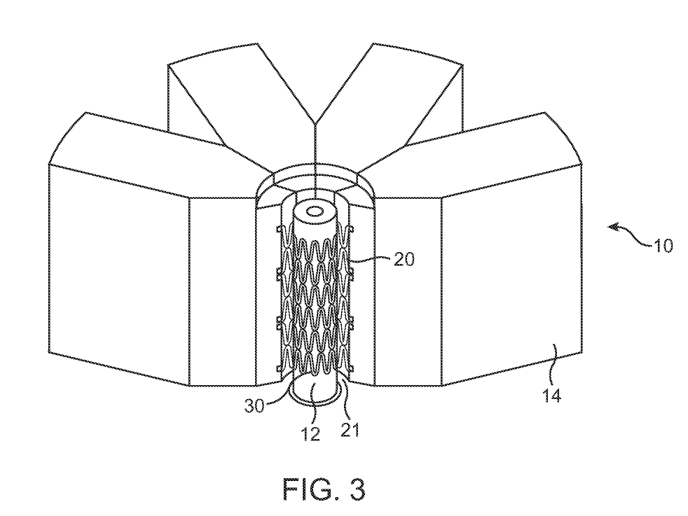

The process for forming a stent using molding system 10 will be described in detail with reference to FIGS. 1-3. FIG. 2 is a partial cut-away view of the molding system 10 shown in FIG. 1. As in FIG. 1, slides 14 of FIG. 2 are in the closed position. As can be seen in FIG. 2, grooves 20 are formed on an inner surface 21 of slides 14. Grooves 20 of each slide match up with the grooves in an adjacent slide so that the overall structure of the stent to be formed is represented by the grooves. For example, in FIG. 1, eight (8) slides 14 are shown, thus, each slide 14 includes grooves 20 forming one-eighth (⅛) of the overall stent structure. However, a different number of slides could be used, for example, four (4) or six (6) in which each of the slides would include grooves 20 forming one-fourth (¼) or one-sixth (⅙) of the overall stent structure. A melted polymer material is injected into grooves 20 through gates 24 to fill grooves 20 with melt. After grooves 20 have been filled with melt, the melt solidifies into the shape of the stent. The slides 14 are moved in the direction of arrows 26 to the open position shown in FIG. 3 and stent 30 is removed. As shown in FIG. 3 the stent pattern is formed by grooves 20 on the inner surface 21 of slides 14. Once stent 30 is removed, the slides can be moved back to the closed position to form another stent.

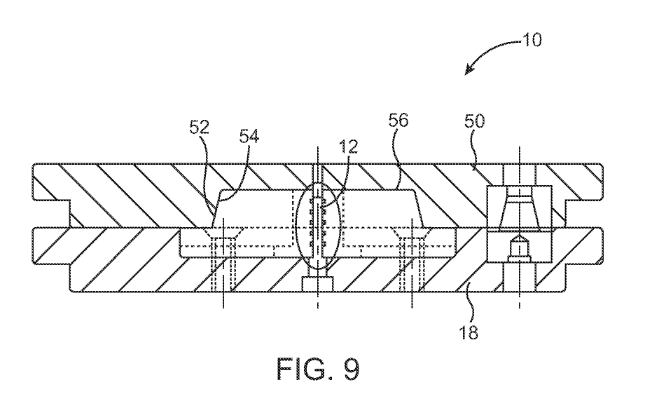

FIG. 9 shows a cross-sectional view of the injection molding system 10 of FIGS. 1-3, further showing a top plate 50 of the injection molding system 10. Top plate 50 is disposed opposite base 14 and includes a cut-out or recess 56. Recess 56 is shaped to fit over slides 14. Further, recess 56 includes slanted side walls 52 which are shaped to correspond to slanted outside walls 54 of slides 14. Thus, when slides 14 are in the open position, top plate 14 is placed over slides 14. Side walls 52 of top plate 14 engage outside walls 54 of slides 14. As top plate 50 continues to be pushed down over slides 14, side walls 52 of top plate 50 slide down outside walls 54 of slides 14, forcing slides 14 inwardly towards central core pin 12. Thus, the step of sliding the slides 14 towards central core pin 12 (the closed position) is accomplished by placing top plate 50 over the slides 14. Upon removal of top plate 50, slides 14 return to the original, open position, such as by spring action, as would be understood by one of ordinary skill in the art.

FIG. 7 shows a perspective view of a polymer stent 30 made in accordance with the methods described in this disclosure. Stent 30 includes struts 32 and crowns 33. Crowns 33 are the bends in stent 30, and struts 32 are the bars extending between crowns 33. Crowns 33 need not be bends or curves, but could be cross-bars or connectors that connect struts 32 together. Likewise struts 32 need not be straight, but may be curved or designed elements that extend between crowns 33.

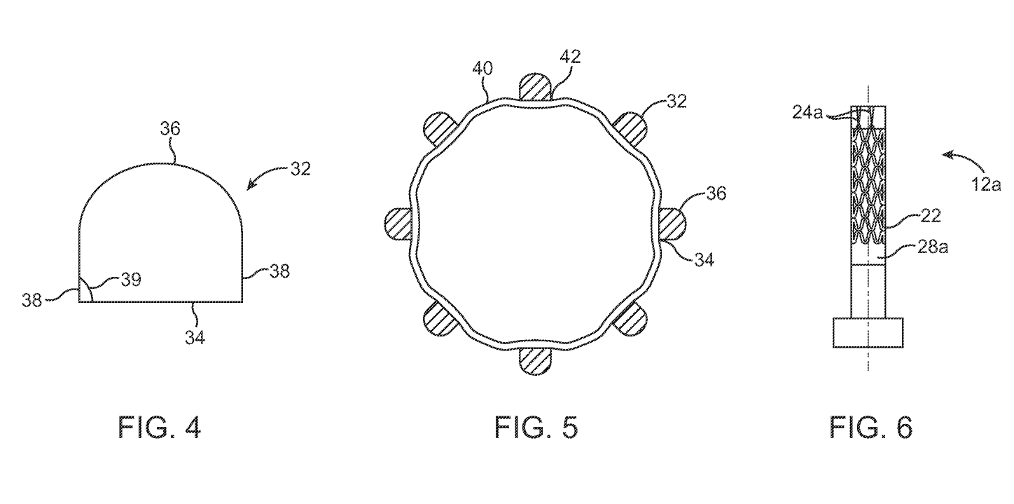

The embodiment shown in FIGS. 2 and 3 shows grooves 20 on inner surface 21 of slides 14, but shows a smooth outer surface 28 for central core pin 12. In such an embodiment, struts 32 and crowns 33 of stent 30 will have a rounded outer surface 36 due to the rounded surface of grooves 20 and will have a relatively flat inner surface 34, as shown in FIG. 4. Such a structure (a “D-shaped”) assists in maintaining stent 30 centered on a balloon during delivery to a treatment site. During insertion of a stent and a balloon through a patient's vasculature, the friction between the exterior of the stent and the blood vessel wall may cause the stent to slip along the outer surface of the balloon, until the stent is no longer properly located at the center of the balloon. This is especially common when the stent is being introduced through a narrow region of a blood vessel. Occasionally, during introduction of the stent, the physician may determine that the lesion to be treated cannot be accessed by the catheter and stent because the vessel is too narrow. The physician must then attempt to withdraw the catheter and stent from the patient's body. In this situation, there is some chance that the stent may slip off the balloon at the distal end of the catheter in an unexpanded state, leaving the unexpanded stent within a blood vessel. D-shaped struts and/or crowns made in accordance with the present disclosure have a relatively flat inner surface 34 with a substantially 90 degree angle 39 between inner surface 34 and side surfaces 38. FIG. 5 shows a cross-section of stent 30 mounted on a balloon 40. Struts 32 include inner surface 34 and outer surface 36. Inner surface 34 includes the surface that comprises the inner diameter of stent 30, which is also the surface of strut 32 that faces inwardly, toward the center of balloon 40. Outer surface 36 of strut 32 includes the surface that is substantially facing away from the center of balloon 40, or may be considered the surface that comprises the outer diameter of strut 32. Because stent 30 of the present invention includes inner surface 34 having angle 39 which is substantially 90 degrees, the sharp edge of strut 32 digs into or grabs the unexpanded surface of balloon 40 while advancing or retracting stent 30 through a patient's vasculature.

After reaching the target site and during expansion, balloon 40 exerts force against inner surface 34 of struts 32. However, because balloon 40 is formed from a thin pliable material, pressure exerted against the exterior of balloon 40 during expansion causes deformation of the exterior of balloon as shown at 42 in FIG. 5. Accordingly, pliable balloon 40 expands slightly around strut 30. Because inner surface 34 of strut 32 includes substantially right angle 39, strut 32 does not easily slide over the surface of balloon 40. Accordingly, strut 32 is held in place during expansion. This aids the physician in properly placing the stent in a patient because the stent will not migrate or slip on the balloon, displacing the stent from its desired position during placement. Further features and advantages of a D-shaped stent are described in U.S. Published Patent Application Publication No. 2003/0187498 A1, which is incorporated in it entirety by reference herein. D-shaped metal stents such as the one described in U.S. Published Patent Application Publication No. 2003/0187498 A1 require complicated tumbling or blasting procedures to form the rounded outer surface of the strut. The method of the present disclosure permits a polymer stent with D-shaped struts without additional steps required to make rounded outer surface 36.

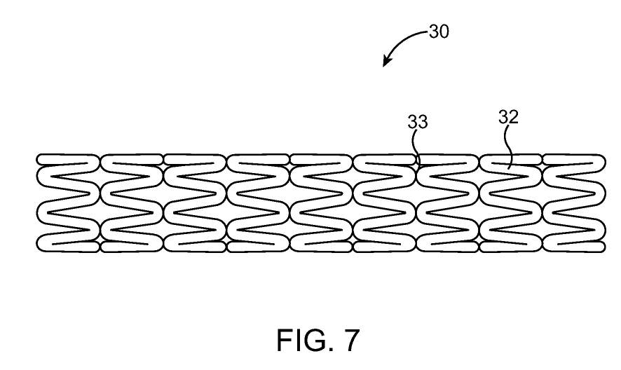

In an alternative embodiment, shown in FIG. 6, central core pin 12a may also have grooves 22 in an outer surface 28a thereof. Such a configuration produces a stent with rounded outer and inner surfaces. Of course, the shape of the grooves in either slides 14 or central core pin 12 is not limited to round or flat. Grooves of various shapes, such as tapered or frustoconical, can be utilized to form various shapes for the outer and inner surfaces of the stent. Gating 24a for melt to enter grooves 22 is also shown in FIG. 6.

The polymer material used to make stent 30 can be any polymer material suitable for use in a human body. Examples of polymers include but are not limited to, poly-a-hydroxy acid esters such as, polylactic acid (PLLA or DLPLA), polyglycolic acid, polylactic-co-glycolic acid (PLGA), polylactic acid-co-caprolactone; poly(block-ethylene oxide-block-lactide-co-glycolide) polymers (PEO-block-PLGA and PEO-block-PLGA-block-PEO); polyethylene glycol and polyethylene oxide, poly(block-ethylene oxide-block-propylene oxide-block-ethylene oxide); polyvinyl pyrrolidone; polyorthoesters; polysaccharides and polysaccharide derivatives such as polyhyaluronic acid, poly (glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclodextrin sulfobutyl ethers; polypeptides and proteins, such as polylysine, polyglutamic acid, albumin; polyanhydrides; polyhydroxy alkonoates such as polyhydroxy valerate, polyhydroxy butyrate, and the like.

Stent 30 can be coated with a therapeutic substance. Further, stent 30 can be formed with recesses or openings in which to deposit such therapeutic substances. Such recesses or openings may be provided by providing indentations at certain locations of grooves 20 or 22. Example of therapeutic substances include, but are not limited to, antineoplastic, antimitotic, antiinflammatory, antiplatelet, anticoagulant, anti fibrin, antithrombin, antiproliferative, antibiotic, antioxidant, and antiallergic substances as well as combinations thereof. Examples of such antineoplastics and/or antimitotics include paclitaxel (e.g., TAXOL® by Bristol-Myers Squibb Co., Stamford, Conn.), docetaxel (e.g., Taxotere® from Aventis S. A., Frankfurt, Germany), methotrexate, azathioprine, vincristine, vinblastine, fluorouracil, doxorubicin hydrochloride (e.g., Adriamycin® from Pharmacia & Upjohn, Peapack N.J.), and mitomycin (e.g., Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax™ (Biogen, Inc., Cambridge, Mass.). Examples of such cytostatic or antiproliferative agents include angiopeptin, angiotensin converting enzyme inhibitors such as captopril (e.g., Capoten® and Capozide® from Bristol-Myers Squibb Co., Stamford, Conn.), cilazapril or lisinopril (e.g., Prinivil® and Prinzide® from Merck & Co., Inc., Whitehouse Station, N.J.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents that may be used include alpha-interferon, genetically engineered epithelial cells, and dexamethasone. In other examples, the therapeutic substance is a radioactive isotope for implantable device usage in radiotherapeutic procedures. Examples of radioactive isotopes include, but are not limited to, phosphoric acid (H₃P³²O₄), palladium (Pd¹⁰³), cesium (Cs¹³¹), and iodine (I¹²⁵). While the preventative and treatment properties of the foregoing therapeutic substances or agents are well-known to those of ordinary skill in the art, the substances or agents are provided by way of example and are not meant to be limiting. Other therapeutic substances are equally applicable for use with the disclosed methods and compositions.

In another embodiment of the present invention, a bioresorbable stent comprises three components mixed together to form a composite. A composite material and engineered materials which consists of more than one material type. A composite is designed to display a combination of the best characteristics of each of the component materials. The three components used in the composite of the present disclosure are a therapeutic substance, a bioresorbable polymer, and a bioresorbable glass fiber or particulate. The therapeutic substance is preferably an antiproliferative drug, such as those listed above. Other therapeutic substances, such as those listed above, may also be used.

The bioresorbable polymer may be any commonly used bioresorbable polymer, for example, poly-L-lactide (PLLA), poly-D,L-lactide (PDLA) and poly-epsilon-caprolactone (PCL). Other bioresorbable polymers, such as those listed above, may be utilized.

The bioresorbable glass fiber or particulate may be, for example, a bioactive hydroxyapatite based glass, such a Bioglass® 45S5, or any other bioresorbable fiber that would improve the mechanical strength of the bioresorbable polymer.

The three components are dissolved in a suitable solvent, such as ethanol, to form a mixture. The solution can then be processed into the desired stent shape by means such as injection molding, spraying, or casting.

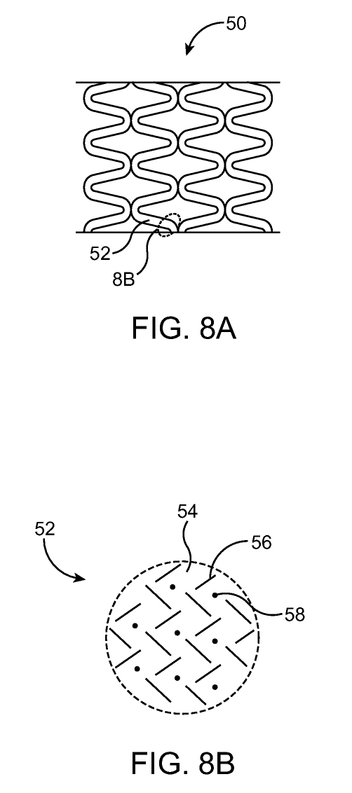

FIG. 8A shows a plan view of a portion of a stent 50 made in accordance with this embodiment. As shown, stent 50 can be shaped similar to stent 30 of FIG. 7. FIG. 8B shows a magnified view a portion of struts 52 of stent 50. As shown in FIG. 8B, struts 52 of stent 50 comprise a polymer 54 with a therapeutic agent 58 and short fiber bioactive glass particles 56 distributed throughout the polymer 54. The polymer 54 acts as a binder agent. As shown, the bioactive glass particles 56 are distributed randomly without a particular orientation.

The composite material used to make stent 50 is advantageous because it has improved mechanical strength over a strictly polymer stent. Accordingly, stents with smaller strut thicknesses are easier to deliver to the site of the lesion and occupy less of the lumen when expanded. The stent 50 made of the composite material comprising a bioerodable polymer, bioactive glass particles, and an antiproliferative drug is advantageous because, among other things, it erodes or dissolves within the vessel after some time, while it erodes or dissolves it distributes the antiproliferative drug to the diseased site, and the bioactive glass particles have some tissue healing properties as they erode or dissolve. Further, due to the semi-elastic properties of the composite used to form the stent, conventional crimping and delivery techniques used for metal stents may be used.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A method of making a stent comprising the steps of:
providing a molding apparatus including a central core pin and a plurality of slides, wherein each of the slides includes grooves on an inner surface the slide, wherein said grooves are formed in the shape of the stent;
injecting a molten polymer into the grooves;
allowing the molten polymer to solidify;
sliding the slides away from the central core pin; and
removing the solidified polymer from the molding apparatus.

2. The method of claim 1, wherein the central core pin includes grooves corresponding to the grooves on the slides, wherein the step of injection a molten polymer into the grooves includes injecting molten polymer into the slide grooves and the central core pin grooves.

3. The method of claim 1, wherein the polymer is selected from the group consisting of poly-a-hydroxy acid esters, poly(block-ethylene oxide-block-lactide-co-glycolide) polymers, polyethylene glycol and polyethylene oxide, poly(block-ethylene oxide-block-propylene oxide-block-ethylene oxide), polyvinyl pyrrolidone, polyorthoesters, polysaccharides and polysaccharide derivatives, polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, polypeptides, polyanhydrides, and polyhydroxy alkonoates.

4. The method of claim 1, wherein the grooves on the inner surface of the slides include indentations configured to form recesses on an outer surface of the stent.

5. The method of claim 4, further comprising the step of adding a therapeutic substance to the recesses formed on the outer surface of the stent.

6. The method of claim 1, further comprising the step of coating the stent with a therapeutic substance.

7. A stent comprising:
struts; and
crowns connecting said struts,
wherein said struts and crowns are formed of a polymeric material, and
wherein said struts have an interior surface and an exterior surface, wherein edges of said interior surface comprise substantially right angles, and wherein edges of said exterior surface are rounded.

8. The stent of claim 7, wherein said struts have a D-shaped cross-section.

9. The stent of claim 7, wherein said crowns include a crown interior surface and a crown exterior surface, wherein edges of said crown interior surface comprise substantially right angles, and wherein edges of said crown exterior surface are rounded.

10. The stent of claim 9, wherein said crowns have a D-shaped cross-section.

11. A stent comprising:
struts, wherein said struts are formed of a composite material comprising a bioerodable polymer, bioactive glass particles, and a therapeutic agent.

12. The stent of claim 11, wherein the bioerodable polymer is selected from the group consisting of poly-L-lactide (PLLA), poly-D,L-lactide (PDLA) and poly-epsilon-caprolactone (PCL).

13. The stent of claim 11, wherein the bioactive glass particles are bioactive hydroxyapatite based glass particles.

14. The stent of claim 11, wherein the therapeutic agent is an antiproliferative agent.
